(19) United States
(12) Patent Application Publication
(10) Pub. No.: US 2020/0261562 A1
(43) Pub. Date: Aug. 20, 2020

(54) COMPOSITION AND METHODS OF MAKING AND USING LEPTOSPIRAL LPS

(71) Applicant: The Regents of the University of California, Oakland, CA (US)

(72) Inventor: Joseph Vinetz, San Diego, CA (US)

(21) Appl. No.: 16/648,844

(22) PCT Filed: Sep. 19, 2018
(86) PCT No.: PCT/US2018/051700

§ 371 Date: Mar. 19, 2020

Related U.S. Application Data
(60) Provisional application No. 62/560,503, filed on Sep. 19, 2017.

(51) Int. Cl. A61K 39/02; C12P 19/04

(52) U.S. Cl. CPC A61K 39/0225; A61K 2039/6037; C12P 19/04

(57) ABSTRACT

The disclosure provides a method to isolate and use, e.g., in a vaccine, high molecular weight lipopolysaccharide, e.g., from Leptospira.

### CROSS REFERENCE TO RELATED APPLICATIONS

This application claims the benefit of the filing date of U.S. application Ser. No. 62/560,503, filed on Sep. 19, 2017, the disclosure of which is incorporated by reference herein.

### STATEMENT REGARDING FEDERALLY SPONSORED RESEARCH OR DEVELOPMENT

This invention was made with government support under Grant No. 1U19AI115658 awarded by the NIH NIAID. The government has certain rights in the invention.

### BACKGROUND

Leptospira is a bacteria that can cause abortion in pregnant mares, kidney and liver failure, and equine recurrent uveitis (ERU, the most common cause of blindness in horses). In dogs and humans it causes severe kidney and/or liver disease, meningitis, respiratory distress and even death without treatment. It is an infection that happens in cattle, pigs, horses, dogs, rodents, wild animals and humans.

The incidence of the disease is increasing in urban dogs as wildlife concentrations increase in these areas. An urban dog is now Just as likely to become infected as a hunting dog. Leptospirosis is treatable with antibiotics when given early in the course of the disease.

All current leptospiral vaccines are bacterins and present leptospiral serovar-specific lipopolysaccharides (LPS) as a T independent antigen.

### SUMMARY

High Molecular Weight (HMW)-LPS, e.g., LPS from about 20 to about 30 kDa that is more than a smear, is very difficult to extract, and for Leptospira, it has not been accomplished so as to yield the predicted laddering with regular polysaccharide, O-antigen subunits found with other Gram-negative bacteria. An improved method for extraction is disclosed herein, which allows for the isolation of HMW-LPS. In one embodiment, HMW-LPS from a spirochete such as Leptospira is conjugated to a carrier protein CRM197), e.g., conjugated to the C) antigen, to convert a T-independent antigen (ag) into a T-dependent ag, which makes it more immunogenic (e.g., allows production of higher affinity and avidity antibodies) and provides longer lasting protection. Thus, a composition is provided having HMW LPS, e.g., from Leptospira, conjugated to a carrier protein such as CRM197 or exotoxin A or any other carrier suitable for increasing immunogenicity. Also provided is a vaccine to prevent, inhibit or treat Leptospirosis in an at or a subject by administering the conjugate. Further provided is a method to extract ITS.

In one embodiment, a method to isolate lipopolysaccharide is provided. The method includes providing a mixture of an isolated culture of a bacteria having lipopolysaccharide subjected to one or more freeze-thaw cycles; heating and/or sonicating the mixture; treating the heated and/or sonicated mixture with one or more nucleases, e.g., one or more non-specific nucleases; treating the nuclease treated mixture with one or more proteases; combining the protease treated mixture with a water-phenol mixture; isolating an aqueous layer and a phenolic layer from the water-phenol treated mixture; optionally dialyzing the aqueous layer and/or dialyzing the phenolic layer; optionally subjecting the aqueous layer and/or the dialyzed phenolic layer to centrifugation so as to provide a pellet having lipopolysaccharide; and isolating and resuspending the pellet. In one embodiment, the aqueous layer is isolated, optionally dialyzed and subjected to centrifugation. In one embodiment, the resuspended pellet is treated with an acid in an amount that removes lipid A from the lipopolysaccharide. In one embodiment, the bacteria is a spirochaete bacteria. In one embodiment, the bacteria comprises Leptospira. In one embodiment, the Leptospira comprises Leptospira interrogans serovars Lai or Copenhageni, or other Leptospira serovars. In one embodiment, the method further comprises treating the resuspended pellet with galactose oxidase and/or sodium periodate. A lipopolysaccharide containing product prepared by the method is further provided. The product may h covalently linked carrier, e.g., a carrier comprising CRM197. In one embodiment, the carrier comprises exotoxin A.

Also provided are vaccine compositions and vaccination methods that prevent or inhibit infection with Leptospira. The vaccine compositions and methods are protective of animals or human subjects. In one embodiment, a method to vaccinate a mammal is provided. In one embodiment, the mammal is a canine. In one embodiment, the mammal is a bovine. In one embodiment, the mammal is an ovine. In one embodiment, the mammal is a swine.

Further provided is a method for producing a vaccine against Leptospirosis. The method includes isolating high molecular weight Leptospiral lipopolysaccharide by a process comprising: providing a mixture of an isolated culture of a bacteria having lipopolysaccharide subjected to one or more freeze-thaw cycles; heating and/or sonicating the mixture; treating the heated and/or sonicated mixture with one or more nucleases; treating the nuclease treated mixture with one or more proteases; combining the protease treated mixture with a water-phenol mixture; isolating an aqueous layer and a phenolic layer from the water-phenol treated mixture; dialyzing the aqueous layer and/or dialyzing the phenolic layer; subjecting the dialyzed aqueous layer and/or the dialyzed phenolic layer to centrifugation so as to provide a pellet having lipopolysaccharide; and isolating and resuspending the pellet; and conjugating Leptospira lipopolysaccharide and a carrier. In one embodiment, the carrier comprises diphtheria toxoid, tetanus toxoid, CRM197, meningococcus outer membrane complex, or Haemophilus protein D.

### BRIEF DESCRIPTION OF THE FIGURES

FIG. 1 shows an exemplary extraction method for LPS.

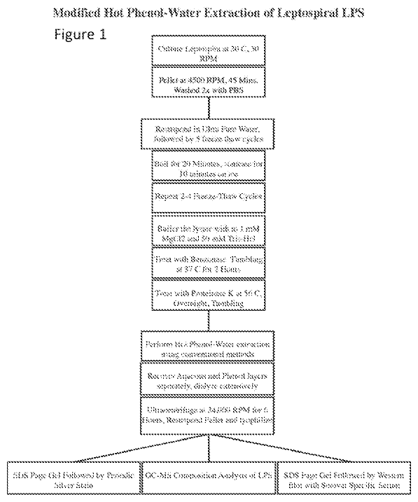

FIG. 2 shows SDS-PAGE/periodate silver stain of purified LPS from different bacteria.

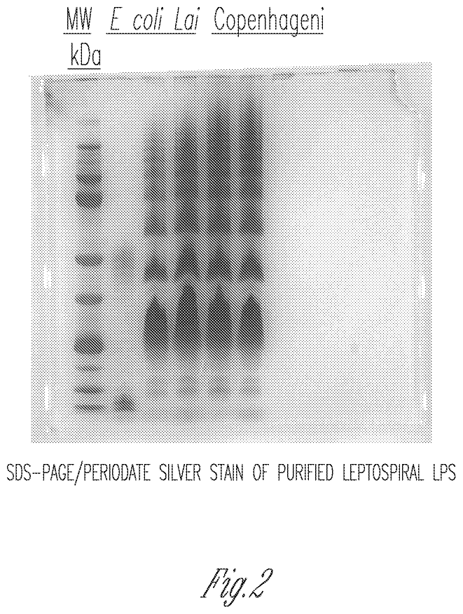

FIGS. 3A-B shows a comparison of staining of LPS from enterobacteria and leptospira generated by a different method (Nally et al., Infection Immunity (2005)). A) Periodate-silver staining of Salmonella rough LPS (2.5 μg) (lane 1), Salmonella smooth LPS (7.5 μg) (lane 2), L. interrogans serovar Copenhageni stain RJ16441 LPS from approximately 7.5×10⁷ cells (lane 3), L. interrogans serovar Copenhageni stain RJ15958 LPS from approximately 7.5×10⁷ cells (lane 4), L. kirsehhneri LPS from approximately 10⁸ cells (lane 5), and L. biflexai LPS from approximately 7.5×10⁷ cells (lane 6). The presence of molecular mass markers (in kilodaltons) are indicated on the left. B) Immunoblot of the same samples as in panel A probed with monoclonal antibody F70C24 specific for LPS of serovar Copenhageni.

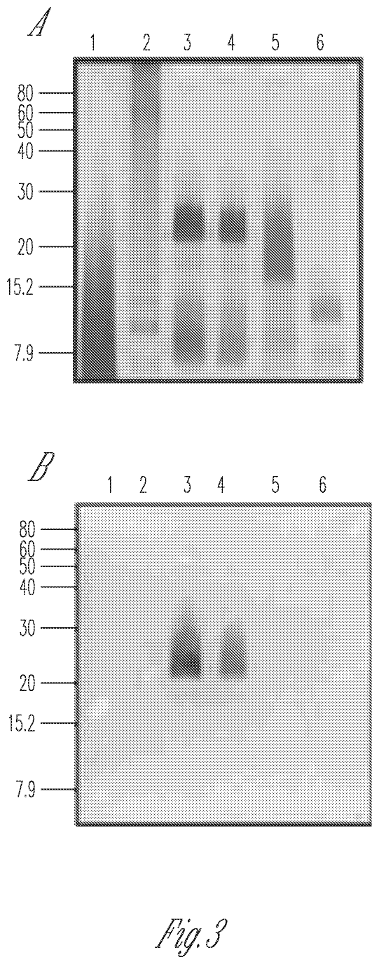

FIG. 4 shows Western blot binding of LPS components by antibodies raised to the Copenhageni strain. 4-12% BT SDS-PAGE gel, MOPS buffer, non-reduced, 125 V, 1×LPS dye. Blocking: 5% non-fat milk; primary: Copenhageni serum raised in rabbit (1:500, 1:2500, and 1:10000); secondary: goat anti-rabbit HRP (1:10000).

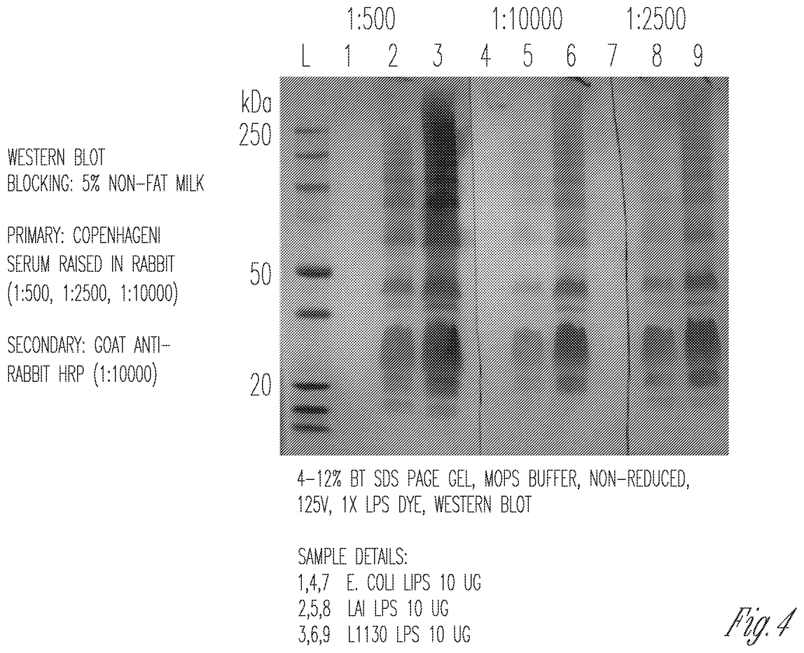

FIG. 5 provides a GCMS composition analysis of oligosaccharides from L130 LPS after mild acid treatment. LPS samples from L130 were hydrolyzed using 2% HOAc in boiling water batch (100° C. for 2.0 h with constant stirring). The samples were cooled in an ice bath and centrifuged at 14,000 RPM, 21,000 g for 20 minutes and the supernatant was lyophilized, then used for composition analysis by GCMS. The sugars labeled in red (see asterisks) were not present in Var-10 (see FIG. 6) and also Rha amount was higher in L130.

FIG. 6 provides a GCMS as TMS derivative analysis of oligosaccharides from Var10 LPS after mild acid treatment. LPS samples from Var-10 were hydrolyzed using 2% HOAc in boiling water batch (100° C. for 2.0 h with constant stirring). The samples were cooled in an ice bath and centrifuged at 14,000 RPM, 21,000 g for 20 minutes and the supernatant was lyophilized, then used for composition analysis by GCMS.

FIG. 7 provides a comparison of the mole percentage of monosaccharides in the oligosaccharides. The total reportable carbohydrate amount is shown in the last column which shows that there is less carbohydrate in Var-10 than L130. LPS associated with virulence has di-deoxy amino sugars and GlcNAc. The other sugars that may contribute to virulence are Rha and Fuc.

FIG. 8 shows a gel with LPS samples including samples subjected to different treatments. 4-12% BT NuPage gel, MOPS buffer, non-reduced, 90 V, 1×LPS dye. Gel visualized under UV. Protein ladder (lane 1); LPS from L1-130 (lane 2), used for conjugation; Acid treated L1-130 LPS (lane 3), SPT containing 0-antigen; Acid treated LPS from L1-130 (lane 4), Lipid A part and pellet Control preconjugation LPS sample (lanes 5-6); ALX-488 conjugated L130-LPS (lanes 7-10); Lipid. A was removed by hydrolysis (lanes 7 and 9) and conjugated to Alx-488; Whole LPS used for conjugation (lanes 8 and 10). Conjugation methods used: oxidation by sodium periodate (Sod. Per. Met) or oxidation by galactose (Gala. Oxid.). AlexaFluor 488-hydrazide is about 570 daltons.

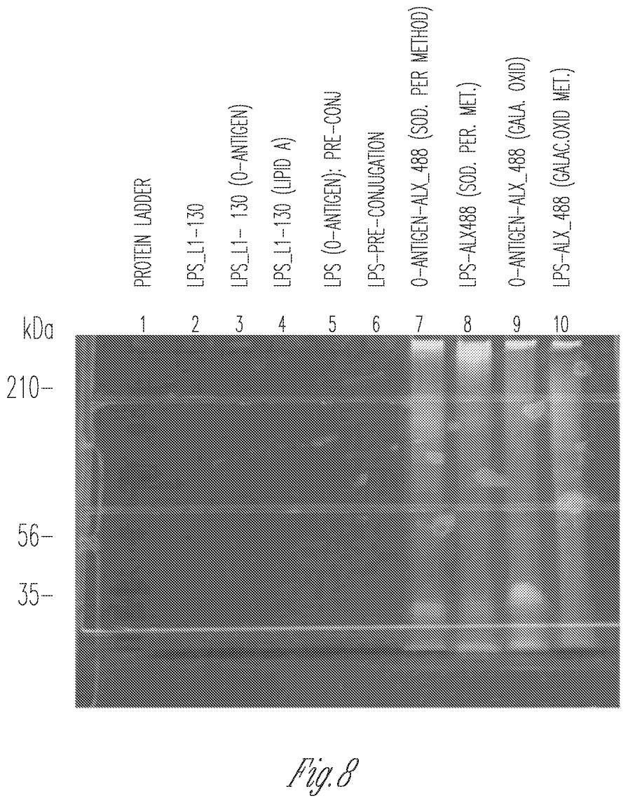

FIG. 9 outlines exemplary experiments.

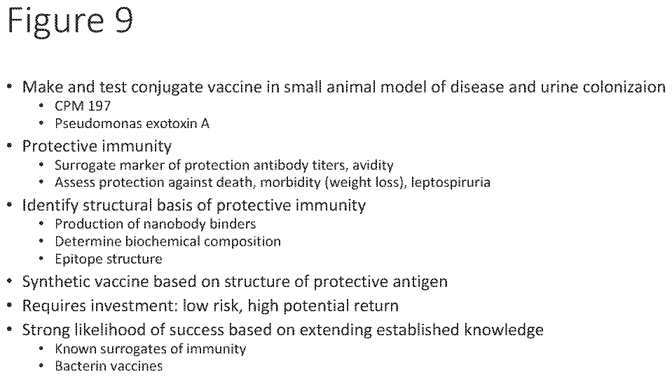

### DETAILED DESCRIPTION

Unlike some of the other major spirochete genera Treponema and Borrelia, the major surface component of Leptospira is LPS. Furthermore, leptospiral LPS is a protective antigen. Monoclonal antibodies against LPS can protect against acute lethal infection in guinea pigs and hamsters (Jost et al. 1986; Schoone et al. 1989) and also protected dogs, based on recovery of leptospires from blood (Schoone et al. 1989). The protection shown in early studies with an “outer sheath” preparation (Aurae: et al. 1972) was almost certainly mediated by LPS. Likewise, it is clear that a reported protective “glycolipid” antigen (Masuzawa et al. 1990) was in fact LPS. Immunization with as little as 2.5 μg of purified LPS, or the polysaccharide (PS) component of LPS, was sufficient to elicit the production of agglutinating, protective antibodies in hamsters (Jost et al. 1989). The immunogenicity of LPS could be enhanced by conjugation with a protein carrier, diphtheria toxoid (Midwinter et al. 1990). An oligosaccharide derived from LPS and conjugated to diphtheria toxoid elicited the production of agglutinating, opsonic antibodies (Midwinter et al. 1994), suggesting that the conjugate would be protective, but protection studies were not undertaken.

The structure of one leptospiral lipid A has been determined (Que-Gewirth et al. 2004), but the structure of the carbohydrate component (O-antigen, core) of leptospiral LPS remains unknown. However, genome sequences have identified LPS biosynthetic loci with close to 100 genes, suggesting that the LPS structure is very complex (Bulach et al. 2006; Nascimento et al. 2004). An intriguing possibility was raised by the use of LPS derived from saprophytic Leptospira billexa to immunize hamsters against infection with the pathogenic serovar Manilae (Matsuo et al. 2000). However, the claims of protection must be tempered by the fact that all hamsters, including controls, survived challenge and developed kidney colonization after a high dose, >10⁶, challenge infection. Protection was based on clinical and pathological criteria, but the use of small animal groups precludes the drawing of any statistically meaningful results. It is possible that the effects observed were due to activation of innate immune responses by L. biflexa LPS. The work has not been reproduced, but significantly, an earlier study in which children were immunized with an inactivated L. biflexa vaccine reported no agglutinating antibodies against pathogenic serovars (Rottini et al, 1972). In addition, another study found no protection against challenge with Canicola in gerbils immunized with L. Biflexa (Sonrier et al. 2000). The clear capacity of LPS and LPS-derived components to elicit protective immunity held the possibility of development of immunoconjugate vaccines, similar to those developed against pneumococcal and Haemophilus influenza infections. However, this development has not eventuated, most probably because of the large number of leptospiral serovars, the cost involved, poor immunogenicity of the carbohydrate (protective) component of the LPS, and the unknown but complex structure of leptospiral LPS, in particular the carbohydrate component.

The disclosure provides purified LPS, leptospiral LPS, including purified LPS conjugated to a carrier, e.g., leptospiral LPS conjugated to a protein, e.g., a carrier protein, and a vaccine, e.g., one that protects a mammal such as hamsters, canines, felines, equines, ovines, caprines, swine, humans, or bovines against death and renal colonization after leptospiral infection. The purified LPS, such as purified leptospiral LPS, conjugated to a protein provides vaccines for the protection of humans and non-human animals.

Compositions of the invention may include one or more of an antimicrobial detergent (e.g., TWEEN (polysorbate), sodium salts (e.g., sodium chloride) a buffer, e.g., a phosphate buffer, a sugar alcohol (e.g., mannitol) or a disaccharide (e.g. sucrose or trehalose), and the pH of a composition may be adjusted. Immunogenic compositions and vaccines of may comprise pharmaceutically acceptable carriers. Suitable carriers are typically large, slowly metabolized macromolecules such as proteins, polysaccharides, polylactic acids, polyglycolic acids, polymeric amino acids, amino acid copolymers, sucrose, trehalose, lactose, lipid aggregates (such as oil droplets or liposomes), and inactive virus particles. Such carriers are well known to those of ordinary skill in the art. The vaccines may also contain diluents, such as water, saline, glycerol. Immunogenic compositions used as vaccines comprise an immunologically effective amount of antigen, as well as any other of the above-mentioned components. By immunologically effective amount, is meant that the administration of that amount to a mammal, either in a single dose or as part of a series, is effective for treatment or prevention. This amount varies depending upon the health and physical condition of the mammal to be treated, age, the taxonomic group to be treated (e.g., non-human primate, human, etc.), the capacity of the mammal's immune system to synthesize antibodies, the degree of protection desired, the formulation of the vaccine, the assessment of the medical situation, and other relevant factors. The amount falls in a relatively broad range that can be determined through routine trials, and a typical quantity per dose may be between 1 μg and 20 μg, about 1 μg to about 2.5 μg, about 4 μg, about 5 μg, or about 10 μg of antigen; between 10 μg and 100 μg, about 10 μg to about 25 μg, about 40 μg, about 50 μg, or about 90 μg of antigen; between 1 mg and 20 mg, about 1 mg to about 2.5 mg, about 4 mg, about 5 mg, or about 10 mg of antigen; or between 10 mg and 200 mg, about 10 mg to about 25 mg, about 40 mg, about 50 mg, or about 100 mg of antigen.

In one embodiment, compositions of the invention are injectable. In one embodiment, compositions of the invention are orally administered. Parenteral injection may be subcutaneous, intraperitoneal, intravenous or intramuscular. Intramuscular administration to the thigh or the upper arm is preferred. Injection may be via a needle (e.g., a hypodermic needle), but needle-free injection may alternatively be used. A typical intramuscular dose value is 0.5 ml. Administration may be a single dose schedule or a multiple dose schedule. Multiple doses may be used in a primary immunization schedule and/or in a booster immunization schedule. A primary dose schedule may be followed by a booster dose schedule. Suitable timing between priming doses (e.g., between 4-16 weeks), and between priming and boosting, can be routinely determined. Thus, the invention provides a method of raising an immune response in a mammal, may comprise a humoral immune response and/or a cellular immune response.

For example, LPS vaccination of hamsters prevents infection with regard to L. interrogans serovar Copenhageni. Vaccines are based on a conjugation of purified L. interrogans serovar Copenhageni to CRM197 carrier protein. Also, monoclonal antibodies against L. interrogans serovar Copenhageni LPS are developed in mice. The function of the mAbs, for example, in terms of binding to live organisms is determined by immunofluorescence, ELISA titers, agglutination titers, and affinity measurements; and protection against challenge infection is determined by passive transfer experiments.

Comparison of mouse polyclonal antibodies against CRM197-conjugated and unconjugated L. interrogans serovar Copenhageni LPS in terms of affinity, avidity and agglutination, and protection against challenge is determined by resistance to infection after passive transfer experiments in which immune sera are compared from the two groups by injection into experimental animals prior to challenge infection, Additional studies include comparison of B cell receptor/CD3 region VDJ diversity usage of B cells after vaccination with CRM197-conjugated and unconjugated L. interrogans serovar Copenhageni LPS and comparing such usage to mAb and polyclonal antibody affinity.

In one embodiment, high molecular weight LPS is obtained from Leptospira grown in culture through a method disclosed herein using Leptospira interrogans serovars Lai and Copenhageni). In one embodiment, the purified LPS is modified by acid hydrolysis to remove Lipid A. In one embodiment, the LPS is modified using galactose oxidase and/or sodium periodate treatment. In one embodiment, the LPS is linked to a protein. In one embodiment, the LPS or modified LPS is covalently linked to a carrier or marker, e.g., AlexaFluor-4188. In one embodiment, the purified leptospiral LPS is conjugated to CRM197 diphtheria toxoid or to pseudomonas exotoxin A so as to result in a T-dependent antigen. In one embodiment, the conjugated LPS is employed as a vaccine, e.g., to prevent, inhibit or treat leptospirosis.

The purified LPS in one embodiment is conjugated to immunogenic carrier proteins that have been already used in human clinical trials and animals vaccinated with the conjugate and subsequently challenged.

The purified LPS has been characterized by SDS-PAGE, Western blot and GC-MS, and is the first high quality LPS produced. Thus, the purified. LPS provides for compositions and methods of using a modified form of the purified LPS to more effectively vaccinate mammals including bovines, pigs, sheep, horses, and dogs, relative to current bacterins, to prevent leptospirosis. The vaccine has fewer side effects, e.g., is safer, and is longer lasting (more potent) than bacterin vaccines. Moreover, conjugating LPS to a carrier protein renders it a T-dependent antigen, which makes it more immunogenic, with higher affinity and avidity antibodies and providing for longer lasting protection.

### REFERENCES

Patra et al., BMC Microbiology, 15:244 (2015).
Westphal and Jann, vol. 5 Bacterial Lipopolysaccharides: extraction with phenol-water and future applications of the procedure, New York: Academic; 1965. New York.

All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

1. A method to isolate lipopolysaccharide, comprising:
a) providing a mixture of an isolated culture of bacteria having lipopolysaccharide subjected to one or more freeze-thaw cycles;
b) heating and/or sonicating the mixture;
c) treating the heated and/or sonicated mixture with one or more nucleases;
d) treating the nuclease treated mixture with one or more proteases;
e) combining the protease treated mixture with a water-phenol mixture;
f) isolating an aqueous layer from the water-phenol treated mixture;
g) dialyzing the aqueous layer;
h) subjecting the dialyzed aqueous layer to centrifugation so as to provide a pellet having lipopolysaccharide; and
i) isolating and resuspending the pellet.

2. The method of claim 1 wherein the resuspended pellet is treated with an acid in an amount that removes lipid A from the lipopolysaccharide.

3. The method of claim 1 wherein bacteria is a spirochaete bacteria.

4. The method of claim 1 wherein the bacteria comprises Leptospira.

5. The method of claim 4 wherein the Leptospira comprises Leptospira interrogans serovars Lai or Copenhageni, or other Leptospira serovars.

6. The method of claim 1 further comprising treating the resuspended pellet with galactose oxidase and/or sodium periodate.

7. A lipopolysaccharide containing product prepared by the method of claim 1.

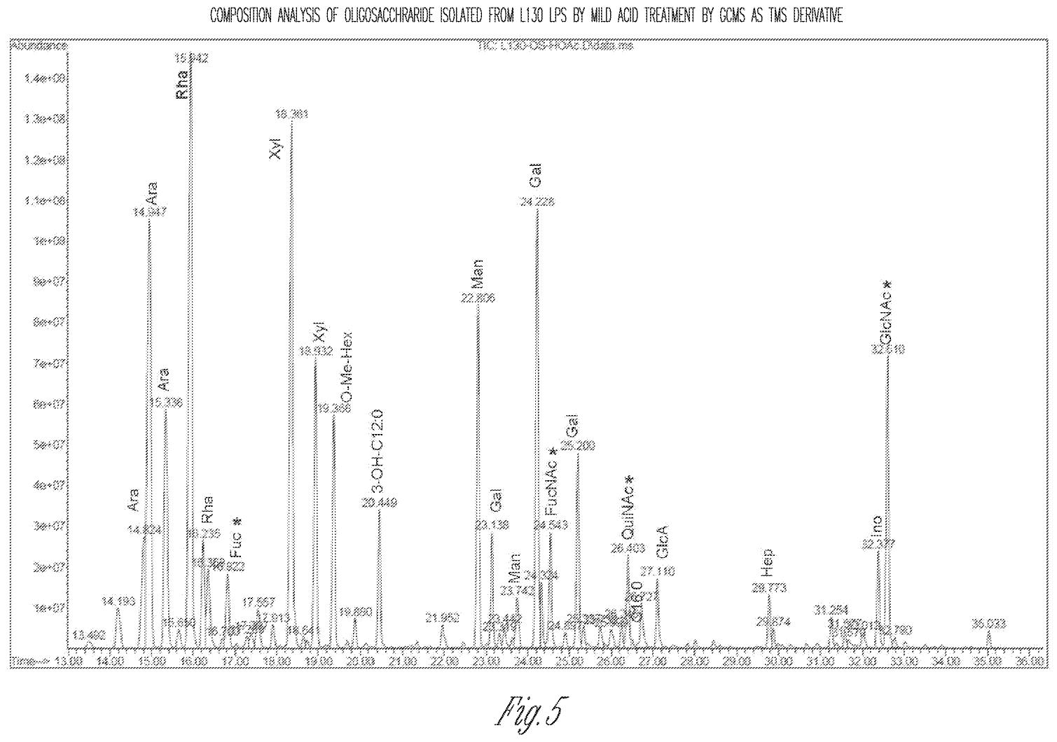

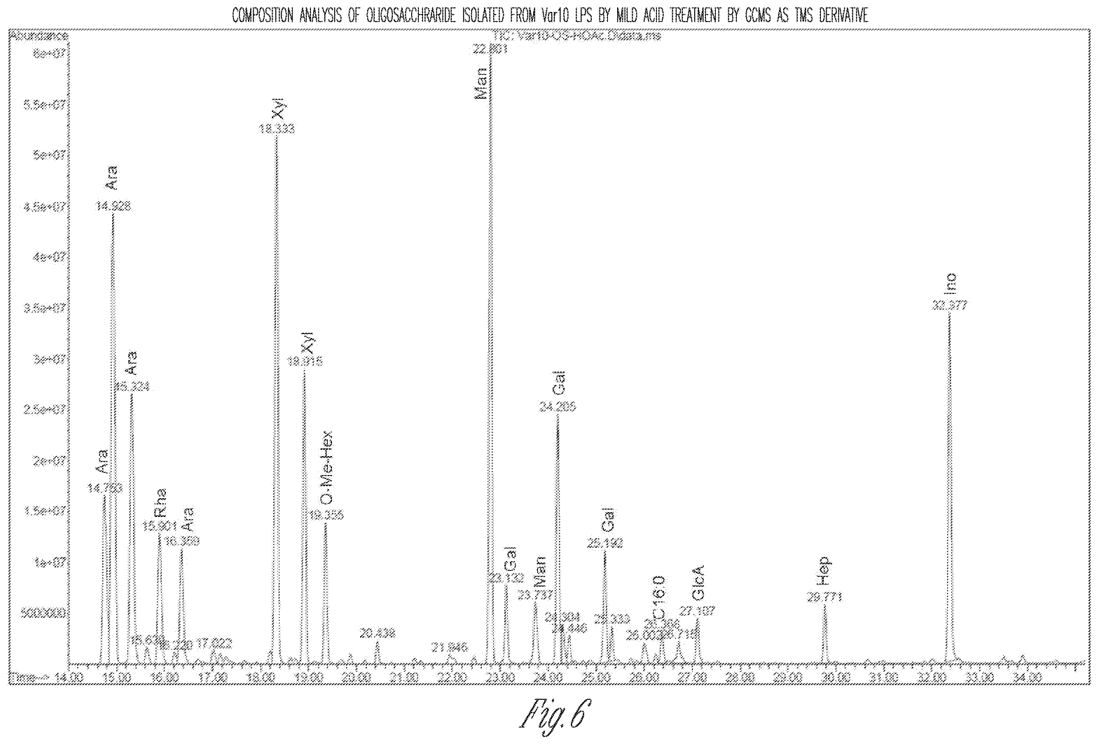

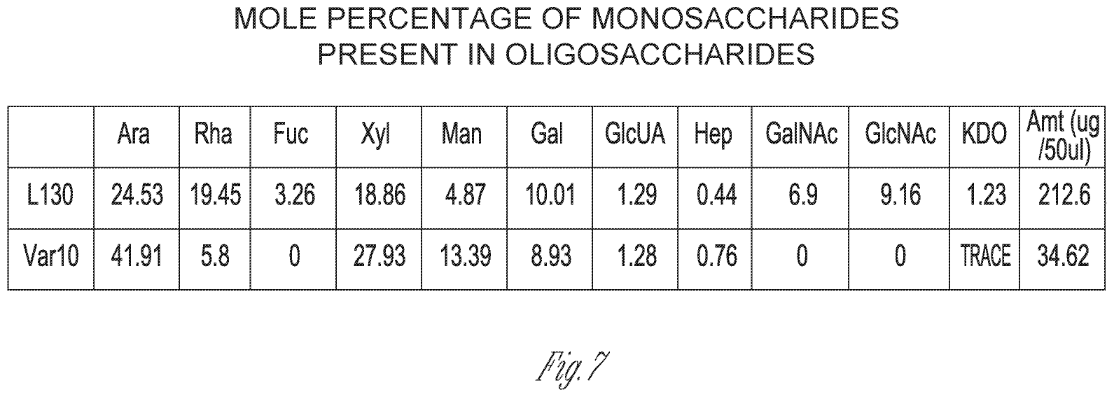

8. The lipopolysaccharide product of claim 7 having the profile in FIGS. 5-7.

9. The lipopolysaccharide product of claim 7 further comprising a covalently linked carrier.

10. The product of claim 9 wherein the carrier comprises CRM197.

11. The product of claim 9 wherein the carrier comprises exotoxin A.

12. A composition comprising a carrier conjugated to high molecular weight Leptospira lipopolysaccharide optionally having reduced lipid A.

13. (canceled)

14. The composition of claim 12 wherein the carrier comprises CRM197.

15. The composition of claim 12 wherein the carrier comprises exotoxin A.

16. The composition of claim 12 which is a vaccine.

17. A method to vaccinate a mammal, comprising administering to a mammal an amount of the composition of claim 12 effective to vaccinate the mammal.

18. The method of claim 17 wherein the mammal is a canine, bovine, ovine, swine or human.

19-26. (canceled)

27. A method for producing a vaccine against Leptospirosis, comprising:
(a) isolating high molecular weight Leptospiral lipopolysaccharide by a process comprising:
i) providing a mixture of an isolated culture of a bacteria having lipopolysaccharide subjected to one or more freeze-thaw cycles;
ii) heating and/or sonicating the mixture;
iii) treating the heated and/or sonicated mixture with one or more nucleases;
iv) treating the nuclease treated mixture with one or more proteases;
v) combining the protease treated mixture with a water-phenol mixture;
vi) isolating an aqueous layer and optionally a phenolic layer from the water-phenol treated mixture;
vii) dialyzing the aqueous layer and optionally dialyzing the phenolic layer;
viii) subjecting the dialyzed aqueous layer and optionally the dialyzed phenolic layer to centrifugation so as to provide a pellet having lipopolysaccharide; and
ix) suspending the pellet comprising Leptospira lipopolysaccharide; and
(b) conjugating the resuspended Leptospira lipopolysaccharide and a carrier.

28. The method of claim 27 wherein the carrier comprises diphtheria toxoid, tetanus toxoid, CRM197, meningococcus outer membrane complex, or Haemophilus protein D.
